(19) United States
(12) Patent Application Publication
(10) Pub. No.: US 2026/0234075 A1
(43) Pub. Date: Aug. 13, 2026

(54) ADDITIVE AND METHOD OF MANUFACTURING SAME

(71) Applicant: Gert Frederick BLOM, Rustenburg (ZA)

(72) Inventor: Gert Frederick BLOM, Rustenburg (ZA)

(21) Appl. No.: 19/160,838

(22) PCT Filed: Feb. 7, 2025
(86) PCT No.: PCT/IB2024/051118

§ 371 Date: Aug. 28, 2025

(30) Foreign Application Priority Data
Feb. 28, 2023 (ZA) 2022/09407

(51) Int. Cl. C05F 9/00; A61L 11/00; C05F 5/00; C05G 5/27

(52) U.S. Cl. CPC C05F 9/00; A61L 11/00; C05F 5/004; C05G 5/27

(57) ABSTRACT

This invention relates to an additive and method of manufacturing same, and more particularly, but not exclusively, to an additive comprising eggshell, and eggshell membrane content. The additive comprises sterilised eggshell and eggshell membrane crushed to have a particle size in the range of 0.4 to 1.6 micron. The additive is dispersed in a liquidized plant-based material such as a liquidized water hyacinth plant (Eichhornia Crassipes).

### FIELD OF THE INVENTION

This invention relates to an additive and method of manufacturing same, and more particularly, but not exclusively, to an additive comprising eggshell, and eggshell membrane content.

### BACKGROUND TO THE INVENTION

Eggshells are waste materials from hatcheries, food industries, and homes and can easily be collected in large amounts.

Eggshells comprises a calcified shell and eggshell membranes which include an inner and outer eggshell membrane. The composition of the eggshell is mainly calcium carbonate, with some trace amounts of other elements. Eggshell membranes comprise a large amount of protein. The eggshell membrane protein consists of collagen as a component.

The minerals and proteins found in eggshell and eggshell membrane are crucial for assisting plant growth.

Eggshells are an effective and inexpensive additive for plants to assist with plant growth.

Two well-known methods exist for preparing eggshell additives. The first method involves crushing the eggshells (“crushing method”) and the second method involves steeping the eggshells (“steeping method”).

In the crushing method the eggshells are washed, rinsed, dried, and crushed to a diameter easily visible to the human eye. The crushed eggshells are placed directly in the root substance of the plants to provide the necessary minerals and proteins to the plant.

The crushing method provides a relatively slow absorption rate by the plant.

In the steeping method the eggshells are boiled in water. They are then left to steep for a certain amount of time before they are strained out. The steeped liquid is poured directly onto the root substance of the plants to provide the necessary minerals and proteins to the plant.

The steeping method only allows for the extraction of a portion of the minerals and proteins from the eggshell. A large portion of the minerals and proteins are lost when the eggshells and eggshell membranes are strained out.

### OBJECT OF THE INVENTION

It is an object of this invention to provide an additive and method of manufacturing which, at least partially, alleviates some of the above-mentioned difficulties.

### SUMMARY OF THE INVENTION

In accordance with this invention there is provided an additive comprising eggshell and eggshell membrane crushed to have a particle size in the range of 0.4 to 1.6 micron.

There is provided for the eggshell and eggshell membrane to be sterilised.

There is further provided for the sterilisation to take place by way of radiation.

There is even further provided for the radiation source to be an ultraviolet light.

There is provided for the additive to be dispersed in a liquid.

There is further provided for the liquid to be a liquidized plant-based material.

There is still further provided for the plant-based material to be a water hyacinth.

There is yet further provided for the water hyacinth to be Eichhornia Crassipes.

There is provided for the plant-based material to be liquidized by means of blending, juicing, or extracting.

According to a second aspect of this invention there is provided a method of manufacturing an additive comprising the steps of:
taking eggshell having its eggshell membrane;
washing the eggshell together with its eggshell membrane;
drying the eggshell together with its eggshell membrane; and
crushing the eggshell together with its eggshell membrane to a particle size in the range of 0.4 to 1.6 micron.

There is provided for the eggshell having its eggshell membrane to be sterilised before being washed.

There is further provided for the sterilisation of the eggshell having its eggshell membrane to take place by way of radiation.

There is even further provided for the radiation means to be an ultraviolet light.

There is provided for the additive to be dispersed in a liquid.

There is further provided for the liquid to be a liquidized plant-based material.

There is still further provided for the plant-based material to be a water hyacinth plant.

There is yet further provided for the water hyacinth plant to be Eichhornia Crassipes.

There is provided for the plant-based material to be liquidized by means of blending, juicing, or extracting.

These and other features of the invention are described in more detail below.

### EXAMPLE OF THE INVENTION

The additive according to the invention was made up by taking eggshells having their eggshell membranes, sterilizing the eggshells having their eggshell membranes, washing the eggshells having their eggshell membranes, drying the eggshells having their eggshell membranes, and crushing the eggshells having their eggshell membranes to the following particle size.

TABLE 1
Crushed particle size of sterilized eggshells
having their eggshell membranes

| | | Size of crushed eggshells |
|---|---|---|
| | | containing eggshell membranes |
| | Description | (micron) |
| 1 | A | 500 < x ≤ 12 000 |
| 2 | B | 100 < x ≤ 500 |
| 3 | C | ≤100 |

The rate of calcium solubility of the prepared samples was experimentally determined over a period of 480 minutes. Results for the experiment are summarised in Table 2.

TABLE 2
Summary of the rate of Calcium Solubility in crushed
eggshells containing eggshell membranes.

| | Size of crushed eggshells | | | Percentage of |
|---|---|---|---|---|
| | containing eggshell | | | Calcium |
| | membranes | Time lapse | | solubility |
| 1.1 | A | 30 | minutes | 40.37% |
| 1.2 | A | 60 | minutes | 40.82% |
| 1.3 | A | 120 | minutes | 41.00% |
| 1.4 | A | 240 | minutes | 41.82% |
| 1.5 | A | 480 | minutes | 41.91% |
| 2.1 | B | 30 | minutes | 35.28% |
| 2.2 | B | 60 | minutes | 35.29% |
| 2.3 | B | 120 | minutes | 35.43% |
| 2.4 | B | 240 | minutes | 36.01% |
| 2.5 | B | 480 | minutes | 36.14% |
| 3.1 | C | 30 | minutes | 39.53% |
| 3.2 | C | 60 | minutes | 40.60% |
| 3.3 | C | 120 | minutes | 40.73% |
| 3.4 | C | 240 | minutes | 42.70% |
| 3.5 | C | 480 | minutes | 42.70% |

### BRIEF DESCRIPTION OF THE DRAWINGS

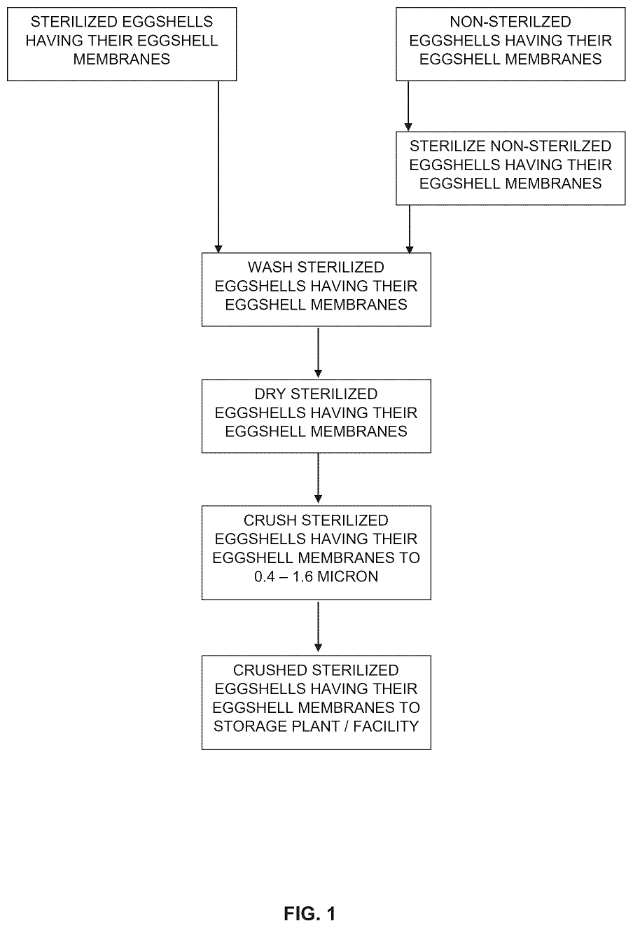

One embodiment of the invention is described below, by way of example only, with reference to the drawing in which FIG. 1 shows a schematic diagram of preparing an additive containing eggshells and eggshell membranes.

### DETAILED DESCRIPTION OF THE DRAWINGS

Sterilised or non-sterilised eggshells containing eggshell membranes are taken from a waste source.

Non-sterilised eggshells containing eggshell membranes are sterilised by way of ultra violet radiation.

The eggshells containing eggshell membranes are washed.

The washed eggshells containing eggshell membranes are dried. The drying means can include, amongst other, by the sun, fans, or through gas heating.

The dried eggshells containing eggshell membranes are crushed to a particle size in the range of 0.4 to 1.6 micron. The crushing means can include, amongst other, rotary crushers, hammer mills, ball mills, rod mills, or cone crushers.

The crushed particles can then be transported to a storage plant or facility.

Additionally, the crushed particles can be dissolved in a liquid such as a plant-based liquid. The plant-based liquid can be a water hyacinth plant that is liquidized using a pulping technique.

It is envisaged that the invention herein described will increase the rate of absorption of the minerals and proteins found in the eggshells containing eggshell membranes by plants and other living organisms. It is further envisaged that the waste eggshells containing eggshell membranes will be utilised in full.

It will be appreciated by those skilled in the art that many other embodiments of the invention are possible without departing from the scope of the invention.

1. An additive comprising eggshell and eggshell membrane crushed to have a particle size in the range of 0.4 to 1.6 micron.

2. An additive as claimed in claim 1, wherein the eggshell and eggshell membrane is sterilised.

3. An additive as claimed in claim 2, wherein the sterilisation takes place by way of radiation.

4. An additive as claimed in claim 3, wherein the radiation comprises ultraviolet light.

5. An additive as claimed in claim 1, wherein the additive is dispersed in a liquid.

6. An additive as claimed in claim 5, wherein the liquid is a liquidized plant-based material.

7. An additive as claimed in claim 6, wherein the plant-based material is a water hyacinth plant.

8. An additive as claimed in claim 7, wherein the water hyacinth plant is Eichhornia Crassipes.

9. An additive as claimed in claim 6, wherein the plant-based material is liquidized using any of the following methods: blending, juicing, or extracting.

10. A method of manufacturing an additive, the method comprising the steps of:
taking eggshell including at least some of its eggshell membrane;
washing the eggshell together with the eggshell membrane;
drying the eggshell together with the eggshell membrane; and
crushing the eggshell and the eggshell membrane to a particle size in the range of 0.4 to 1.6 micron.

11. A method as claimed in claim 10, further including the step of sterilising the eggshell together with the eggshell membrane before washing the eggshell and eggshell membrane.

12. A method as claimed in claim 10, wherein the sterilisation takes place by way of radiation.

13. A method as claimed in claim 12, wherein the radiation comprises ultraviolet light.

14. A method as claimed in claim 10, further including the step of dispersing the additive in a liquid.

15. A method as claimed in claim 14, wherein the liquid is a liquidized plant-based material.

16. A method as claimed in claim 15, wherein the plant-based material is a water hyacinth plant.

17. A method as claimed in claim 16, wherein the water hyacinth plant is Eichhornia Crassipes.

18. A method as claimed in claim 15, wherein the plant-based material is liquidized using any of the following methods: blending, juicing, or extracting.
